(19) United States
(12) Patent Application Publication
(10) Pub. No.: US 2025/0334582 A1
(43) Pub. Date: Oct. 30, 2025

(54) DETECTION METHOD FOR IMMUNE PROTEIN OF HUMAN UMBILICAL CORD MESENCHYMAL STEM CELLS (hUC-MSCs)

(71) Applicant: SCLnow Biotechnology Co., Ltd., Beijing (CN)

(72) Inventors: Cheng Li, Beijing (CN); Lei Guo, Beijing (CN)

(21) Appl. No.: 18/679,260

(22) Filed: May 30, 2024

(30) Foreign Application Priority Data
Apr. 30, 2024 (CN) 2024105413127

(51) Int. Cl. G01N 33/68; C12N 5/0775; C12N 11/00; G01N 1/30

(52) U.S. Cl. CPC G01N 33/68; C12N 5/0668; C12N 11/00; G01N 1/30

(57) ABSTRACT

A detection method for immune protein of human umbilical cord mesenchymal stem cells (hUC-MSCs) may include steps of inoculating hUC-MSCs in a 24-well plate, and a medium is added for incubation. When the hUC-MSCs grow to 60%-80%, the medium is discarded, immobilization is conducted, and immobilized cells are obtained. The immobilized cells obtained is mixed with a substrate application solution for incubation away from light, and mixed with a re-staining solution for staining, and microscopic observation is conducted.

### FIELD OF THE INVENTION

The present invention relates to the technical field of stem cell detection, and particularly relates to a detection method for immune protein of human umbilical cord mesenchymal stem cells (hUC-MSCs).

### BACKGROUND OF THE INVENTION

Mesenchymal stromal/stem cells (MSCs) are a kind of adult stem cells other than hematopoietic stem cells that exist in a variety of tissues (such as bone marrow, umbilical cord blood, umbilical cord tissue, placenta tissue and adipose tissue) and have a multi-directional differentiation potential. This kind of stem cells have a potential to differentiate into a variety of mesenchymal series cells (such as osteoblasts, chondroblasts and lipoblasts) or non-mesenchymal series cells, and have a unique cytokine secretion function.

Multipotency of MSCs means an ability to form more than one type of cells in organisms, which can induce differentiation into a variety of functional cells such as osteocytes and adipocytes in vitro, and can express genetic characteristics that regulate cell differentiation into other histiocyte types or organs (for example, the genes of SSEA-4, OCT-4, SOX-2 and NANOG are multipotent genes). MSCs have a characteristic of producing new cells by cell division, including a feature that the proportion of different stages in a cell cycle and the number of cells during cell division are changed over time. Immune protein of hUC-MSCs needs to be detected in order to control the quality requirements of MSCs.

### SUMMARY OF THE INVENTION

To solve the above problems, the present invention provides a detection method for immune protein of human umbilical cord mesenchymal stem cells (hUC-MSCs).

To achieve the above purpose, the present invention provides the following technical solution: The present invention provides a detection method for immune protein of human umbilical cord mesenchymal stem cells (hUC-MSCs), comprising the following steps:
1) Inoculating hUC-MSCs in a 24-well plate, and adding a medium for incubation;
2) When the hUC-MSCs grow to 60%-80%, discarding the medium, conducting immobilization, and obtaining immobilized cells;
3) Adding a substrate application solution (a mixture of matrix solution A and matrix solution B in an AKP staining kit) into the immobilized cells obtained in step 2) for incubation away from light, mixing the immobilized cells with a re-staining solution (hematoxylin buffer solution) for staining, and conducting microscopic observation;

When no brown or dark-brown particle is found in cytoplasm, no immune protein is secreted by the hUC-MSCs;

When brown or dark-brown particles are found in cytoplasm, immune protein is secreted by the hUC-MSCs;

Preferably, the inoculum concentration of the hUC-MSCs in step 1) is 1×10⁵ cells/well.

Preferably, the hUC-MSCs in step 1) belong to any generation from P3 to P6.

Preferably, the medium in step 1) is a serum-free medium, with an addition amount of 1 mL per well.

Preferably, the conditions of the incubation in step 1) include: the incubation is conducted at 37° C. and 5% CO₂.

Preferably, the immobilization in step 2) is conducted using a paraformaldehyde solution with a mass percentage of 4%, and the time is 4 min.

Preferably, the addition amount of the substrate application solution in step 3) is 200 μl/well. Preferably, the addition amount of the re-staining solution in step 3) is 200 μl/well.

Preferably, the conditions of the incubation away from light in step 3) include: the temperature is 20-25° C., and the time is 15 min.

Preferably, the time for the staining in step 3) is 3 min.

The present invention has the following beneficial effects:

The present invention uses alkaline phosphatase (AKP) in cells to hydrolyze naphthol phosphate in an alkaline environment to generate naphthol, and the latter is coupled with a diazosalt to generate an insoluble colored precipitate which is anchored to a zymophore in cytoplasm. The method has the advantages of simple operation, high sensitivity and easy access to instruments required.

### DESCRIPTION OF DRAWINGS

To more clearly describe the technical solution in the embodiments of the present invention or in the prior art, the drawings required to be used in the embodiments will be simply presented below.

FIG. 1 is a schematic diagram showing a positive result of alkaline phosphatase detection in hUC-MSCs.

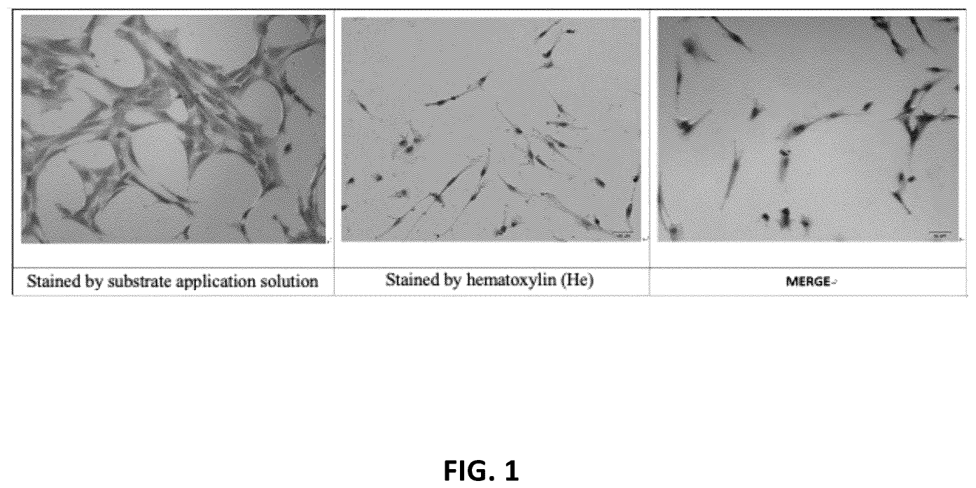

### DETAILED DESCRIPTION OF THE INVENTION

The present invention provides a detection method for immune protein of human umbilical cord mesenchymal stem cells (hUC-MSCs), comprising the following steps:
1) Inoculating hUC-MSCs in a 24-well plate, and adding a medium for incubation;
2) When the hUC-MSCs grow to 60%-80%, discarding the medium, conducting immobilization, and obtaining immobilized cells;
3) Adding a substrate application solution (a mixture of matrix solution A and matrix solution B in an AKP staining kit) into the immobilized cells obtained in step 2) for incubation away from light, mixing the immobilized cells with a re-staining solution (hematoxylin buffer solution) for staining, and conducting microscopic observation;

When no brown or dark-brown particle is found in cytoplasm, no immune protein is secreted by the hUC-MSCs;

When brown or dark-brown particles are found in cytoplasm, immune protein is secreted by the hUC-MSCs;

According to the present invention, hUC-MSCs are inoculated in a 24-well plate, and a medium is added for incubation. In the present invention, the inoculum concentration of the hUC-MSCs is preferably 1×10⁵ cells/well. In the present invention, the hUC-MSCs preferably belong to any generation from P3 to P6. In the present invention, the medium is preferably a serum-free medium, with an addition amount of preferably 1 mL per well. In the present invention, the conditions of the incubation include: the incubation is conducted at 37° C. and 5% CO₂.

According to the present invention, when the hUC-MSCs grow to 60%-80%, the medium is discarded, immobilization is conducted, and immobilized cells are obtained. In the present invention, the immobilization is conducted using a paraformaldehyde solution with a mass percentage of preferably 4%, and the time is preferably 4 min.

According to the present invention, a substrate application solution (a mixture of matrix solution A and matrix solution B in an AKP staining kit) is added into the immobilized cells obtained for incubation away from light, the immobilized cells is mixed with a re-staining solution (hematoxylin buffer solution) for staining, and microscopic observation is conducted; when no brown or dark-brown particle is found in cytoplasm, no immune protein is secreted by the hUC-MSCs; when brown or dark-brown particles are found in cytoplasm, immune protein is secreted by the hUC-MSCs; in the present invention, the addition amount of the substrate application solution is preferably 200 μl/well. In the present invention, the addition amount of the re-staining solution is preferably 200 μl/well. In the present invention, the conditions of the incubation away from light preferably include: the temperature is 20-25° C., and the time is 15 min. In the present invention, the time of the staining is preferably 3 min.

To further describe the present invention, the prevent invention will be described below in detail in combination with embodiments, but the embodiments shall not be understood as the limitation of the protection scope of the present invention.

### Embodiment 1

### 1. Method and Principle

AKP is used to hydrolyze naphthol phosphate in an alkaline solution (pH 9.0-9.8) to generate naphthol, and the latter is coupled with a diazosalt to generate an insoluble colored precipitate which is anchored to a zymophore in cytoplasm.

Multipotent stem cells can express AKP at a high level, but once the cells are differentiated, expression level of AKP will be decreased significantly. Therefore, AKP staining is an effective means to detect whether stem cells are differentiated.

### 2. Reagents

Sodium chloride (NaCl): analytical reagent; sodium hydrogen phosphate (Na₂HPO₄·12H₂O): analytical reagent; potassium chloride (KCl): analytical reagent; potassium dihydrogen phosphate (KH₂PO₄): analytical reagent; paraformaldehyde: analytical reagent; PBS buffer solution: 8.0 g of NaCl, 2.9 g of Na₂HPO₄·12H₂O, 0.2 g of KCl and 0.2 g of KH₂PO₄ are weighed respectively, dissolved with water, diluted to 1000 mL and subjected to autoclaved sterilization for later use after split charging; 4% paraformaldehyde: 4 g of paraformaldehyde is weighed, added with 100 mL of PBS buffer solution, placed in a water bath at 60° C. overnight for dissolving, and preserved at 4° C. for later use; an AKP staining kit (D001 from Nanjing Jiancheng Bioengineering Institute): fibroblast negative control.

### 3. Materials

A 24-well cell culture plate: made of clinical-grade polypropylene material, sterile, non-pyrogenic and DNase/RNase free; centrifugal tubes, pipettes and tips: sterile, non-pyrogenic and DNase/RNase free; aluminum foil.

### 4. Instruments

Pipettes: with maximum ranges of 200 μL/1000 μL; an electric continuous dispenser: suitable for 5 mL/10 mL pipettes; a low speed freezing centrifuge: with a maximum centrifugal speed of 4500 r/min; an inverted microscope; a balance: with a reciprocal sensibility of 0.1 mg; a water bath; a constant temperature and humidity CO₂ incubator.

### 5. Testing

### 5.1 Cell Culture

SCLnow hUC-MSCs of P3-P6 generations are inoculated into the 24-well plate at a density of 1×10⁵ cells/well, 1 mL of serum-free medium is added into each well, the plate is placed in the incubator with 5% CO₂ at 37° C. for incubation and observed by the microscope, and staining can be conducted when the cells grow to 60%-80%.

### 5.2 Sample Testing

The medium is gently sucked out by a pipette and discarded; 1 mL of PBS is added to wash the cells for 2 to 3 times, and the washing solution is sucked out and discarded; 200 μL of 4% paraformaldehyde is added to immobilize the cells for 10 min, the 4% immobilizing solution is sucked out and discarded, and the cells are rinsed with PBS for 2 to 3 times; after sample immobilization, 200 μL of freshly prepared substrate application solution is dripped into each well according to the instructions of the AKP staining kit, and incubation is conducted away from light at room temperature for 15 min; the cells are rinsed with PBS for 2 to 3 times, 10 min each time, and the rinsing solution is sucked out and discarded; the cells are stained while wet with 200 μL of re-staining solution for 3 min; the cells are washed with ultra-pure water for 2 to 3 times to finish a staining reaction, and staining results are observed under the inverted microscope.

### 5.3 Result Determination

Negative: no brown or dark-brown particle is found in cytoplasm;
Weakly positive: brown or dark-brown particles are occasionally or slightly found in cytoplasm;
Positive: significant dark-brown or brown or black particles, chunks or strip-shaped precipitate are found in cytoplasm;

The present invention is described in detail by the above embodiments. However, the above embodiments are merely part of the embodiments of the present invention, not all of the embodiments, and other embodiments can be obtained based on the embodiments in the present invention without contributing creative labor, which will also belong to the protection scope of the present invention.

What is claimed is:

1. A detection method for immune protein of human umbilical cord mesenchymal stem cells (hUC-MSCs), comprising the following steps:
1) inoculating hUC-MSCs in a 24-well plate, and adding a medium for incubation;
2) when the hUC-MSCs grow to 60%-80%, discarding the medium, conducting immobilization, and obtaining immobilized cells;
3) adding a substrate application solution into the immobilized cells obtained in step 2) for incubation away from light, mixing the immobilized cells with a re-staining solution for staining, and conducting microscopic observation; when no brown or dark-brown particle is found in cytoplasm, no immune protein is secreted by the hUC-MSCs; and when brown or dark-brown particles are found in cytoplasm, immune protein is secreted by the hUC-MSCs.

2. The detection method according to claim 1, wherein the inoculum concentration of the hUC-MSCs in step 1) is 1×10⁵ cells/well.

3. The detection method according to claim 1, wherein the hUC-MSCs in step 1) belong to any generation from P3 to P6.

4. The detection method according to claim 1, wherein the medium in step 1) is a serum-free medium, with an addition amount of 1 mL per well.

5. The detection method according to claim 1, wherein the conditions of the incubation in step 1) include: the incubation is conducted at 37° C. and 5% CO₂.

6. The detection method according to claim 1, wherein the immobilization in step 2) is conducted using a paraformaldehyde solution with a mass percentage of 4%, and the time is 4 min.

7. The detection method according to claim 1, wherein the addition amount of the substrate application solution in step 3) is 200 μl/well.

8. The detection method according to claim 1, wherein the addition amount of the re-staining solution in step 3) is 200 μl/well.

9. The detection method according to claim 1, wherein the conditions of the incubation away from light in step 3) include: the temperature is 20-25° C., and the time is 15 min.

10. The detection method according to claim 1, wherein the time for the staining in step 3) is 3 min.
